(19) United States
(12) Patent Application Publication
(10) Pub. No.: US 2021/0403964 A1
(43) Pub. Date: Dec. 30, 2021

(54) CANNABINOID PRODUCTION IN ALGAE

(71) Applicant: ENZYMOGEN LTD, Yavne (IL)

(72) Inventor: Avraham LABAN, Jerusalem (IL)

(73) Assignee: ENZYMOGEN LTD, Yavne (IL)

(21) Appl. No.: 17/048,794

(22) PCT Filed: Apr. 16, 2019
(86) PCT No.: PCT/IB2019/053139

§ 371 Date: Oct. 19, 2020

Related U.S. Application Data
(60) Provisional application No. 62/658,617, filed on Apr. 17, 2018.

(51) Int. Cl. C12P 17/06; C12N 1/12; C12N 15/82; C12N 9/02; C12N 9/88

(52) U.S. Cl. CPC C12P 17/06; C12N 1/12; C12R 2001/89; C12N 9/0004; C12N 9/88; C12N 15/8243

(57) ABSTRACT

An expression system and method for producing a cannabinoid in algae are provided. The method includes expressing in an algae cell an enzyme for converting hexanoic acid to hexanoyl-CoA, enzymes for converting hexanoyl-CoA to olivetolic acid (OA), an enzyme for converting olivetolic acid (OA) to cannabigerolic acid (CbGA) and an enzyme for converting cannabigerolic acid (CbGA) to a cannabinoid.

### RELATED APPLICATION

This is the U.S. National Stage of International Patent Application No. PCT/IB2019/053139 filed on Apr. 16, 2019, which in turn This application claims the benefit of priority of U.S. Provisional Patent Application No. 65/628,617 filed on Apr. 17, 2018, the contents of which are incorporated herein by reference in their entirety.

### SEQUENCE LISTING

The nucleic acid sequences provided herewith are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file named 96239_303_1_seq_ST25, created Mar. 14, 2021, about 21.4 KB, which is incorporated by reference herein.

### BACKGROUND

The present invention relates to an expression system and a method of using same for producing a cannabinoid in algae. Embodiments of the present invention relate to an expression system capable of converting a fatty acid such as hexanoic acid into a cannabinoid in algae and to methods of producing a cannabinoid in, and purifying a cannabinoid from an algae culture.

Cannabinoids are terpenophenolic compounds produced naturally in mammals and plants (phytocannabinoids) such as Cannabis sativa and hemp. The most active of the naturally occurring cannabinoids is tetrahydrocannabinol (THC). THC is used for treating a wide range of medical conditions, including glaucoma, AIDS wasting, neuropathic pain, treatment of spasticity associated with multiple sclerosis, fibromyalgia and chemotherapy-induced nausea. Additional active cannabinoids include cannabidiol (CBD), an isomer of THC, which is a potent antioxidant and anti-inflammatory and Cannabigerol (CBG); both CBD and CBG are abundant in hemp species.

Cannabinoids are typically extracted from plants or produced via chemical synthesis. Plant extraction does not yield a pure product as reflected by the fact that the FDA has yet to approve a cannabinoid product extracted from plants. Extraction of cannabinoids from Cannabis sativa is carried out by placing the plant in a chemical solution that selectively solubilizes the cannabinoids. The cannabinoid-containing solution is then further processed to produce a partially purified cannabinoid extract. Chemical synthesis is a complex and costly process but produces a highly purified cannabinoid product.

Algae are an ideal platform for large-scale production of chemical products since they are fast-growing and can be grown in solar-powered bio-factories with minimal nutrient requirements. Algae are eukaryotes (blue algae, the exception, is a prokaryote), which, unlike bacteria, efficiently produce complex proteins and contain the machinery necessary to fold and assemble multi-component complexes into functional proteins. In addition, green algae are generally regarded as safe (GRAS), and therefore pose little risk of viral, prion or bacterial endotoxin contamination.

Algae propagate by vegetative replication, lack pollen, and have no potential for gene transfer to food crops. They can easily be grown in containment, thus reducing any chance of environmental contamination or contamination of the produced product by external contaminants such as pesticides or pollutants.

Thus, it would be highly advantageous to have a genetically modified algae capable of producing a cannabinoid.

### SUMMARY

According to one aspect of the present invention there is provided a method of producing a cannabinoid in algae comprising expressing in an algae cell: an enzyme for converting hexanoic acid to hexanoyl-CoA; enzymes for converting hexanoyl-CoA to olivetolic acid (OA); an enzyme for converting olivetolic acid (OA) to cannabigerolic acid (CbGA); and an enzyme for converting cannabigerolic acid (CbGA) to a cannabinoid.

According to embodiments of the present invention the algae cell is transformed with polynucleotide sequences encoding Hexanoyl synthase, Prenyl synthase, Olivetolic acid cyclase and Prenyl transferase.

According to embodiments of the present invention the algae cell is grown in a basic growth medium.

According to embodiments of the present invention the algae cell is grown in a growth medium supplemented with hexanoic acid.

According to embodiments of the present invention the algae cell is grown in a growth medium supplemented with olivetolic acid.

According to embodiments of the present invention a polynucleotide sequence of the Hexanoyl synthase, Prenyl synthase, Olivetolic acid cyclase and/or Prenyl transferase is expressed from an inducible promoter.

According to embodiments of the present invention the inducible promoter is induced by galactose.

According to embodiments of the present invention the inducible promoter is induced by IPTG.

According to embodiments of the present invention the inducible promoter is induced by Heat shock.

According to embodiments of the present invention the inducible promoter is induced by light.

According to embodiments of the present invention the inducible promoter is induced by tetracycline.

According to embodiments of the present invention the method further comprises recovering the cannabinoid from the growth medium.

According to embodiments of the present invention the algae cell further expresses: an enzyme for converting the cannabinoid to cannabidiol (CBD) or tetrahydrocannabinol (THC).

According to embodiments of the present invention the algae cell is further transformed with polynucleotide sequences encoding cannabidiolic acid synthase or THC synthase.

According to embodiments of the present invention a sequence of the polynucleotide sequences is optimized for expression in an algae cell.

According to embodiments of the present invention the algae cell is selected from the group consisting of Green algae, red algae, Euglenids, Chromista, Dinoflagellates and Cyanobacteria.

According to embodiments of the present invention the algae cell is selected from the group consisting of Chlorophytes, Charophyta and Rhodophyta.

According to another aspect of the present invention there is provided an expression system for expression in an algae cell comprising polynucleotide sequences encoding Hexanoyl synthase, Prenyl synthase, Olivetolic acid cyclase and Prenyl transferase under the control of an inducible promoter.

According to embodiments of the present invention the inducible promoter is induced by galactose.

According to embodiments of the present invention the inducible promoter is induced by IPTG.

According to embodiments of the present invention the inducible promoter is induced by Heat shock.

According to embodiments of the present invention the inducible promoter is induced by light.

According to embodiments of the present invention the inducible promoter is induced by tetracycline.

According to embodiments of the present invention a sequence of the polynucleotide sequences is optimized for expression in an algae cell.

According to embodiments of the present invention the algae cell is selected from the group consisting of Green algae, Red algae, Euglenids, Chromista, Dinoflagellates and Cyanobacteria.

According to embodiments of the present invention the algae cell is selected from the group consisting of Chlorophytes, Charophyta and Rhodophyta.

According to embodiments of the present invention each of the polynucleotide sequences includes at least one intron.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIG. 1 is a prior art drawing of the synthesis pathway of cannabinoids in Cannabis sativa.

FIGS. 2A-3D schematically illustrate plasmid constructs utilized by the present invention.

FIG. 4 illustrates the HPLC peaks of various compounds produced by the method of the present invention.

FIG. 5 is Table showing HPLC results for olivetolic acid and CBGA for various clones produced by the method of the present invention.

### DETAILED DESCRIPTION

The present invention is of an expression system which can be used to produce a cannabinoid in algae. Specifically, the present invention can be used to produce large amounts of highly purified cannabinoids in an algae culture.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Research has shown that cannabinoids can be useful for the treatment of various medical conditions. Treatment requires a purified active agent which, at present, can only be produced using chemical synthesis approaches. While extraction of cannabinoids from plants is more cost effective it does not yield a purified product that can be used as an API in pharmaceutical preparations and has to be further purified.

Since chemical synthesis of cannabinoids is costly and complicated, research has turned to cell cultures in an attempt to produce a highly purified and concentrated cannabinoid product that can be used as an API.

Both prokaryotic and eukaryotic culture systems have been investigated with limited success.

Bacterial and yeast expression systems used for heterologous production of cannabinoids were typically selected based on their suitability for genetic manipulation. The absence of significant amounts of essential building blocks limited the production of cannabinoids to very small amounts in bacteria and yeast and as such, extensive manipulation of metabolic pathways was required in order to produce detectable amounts of CBG.

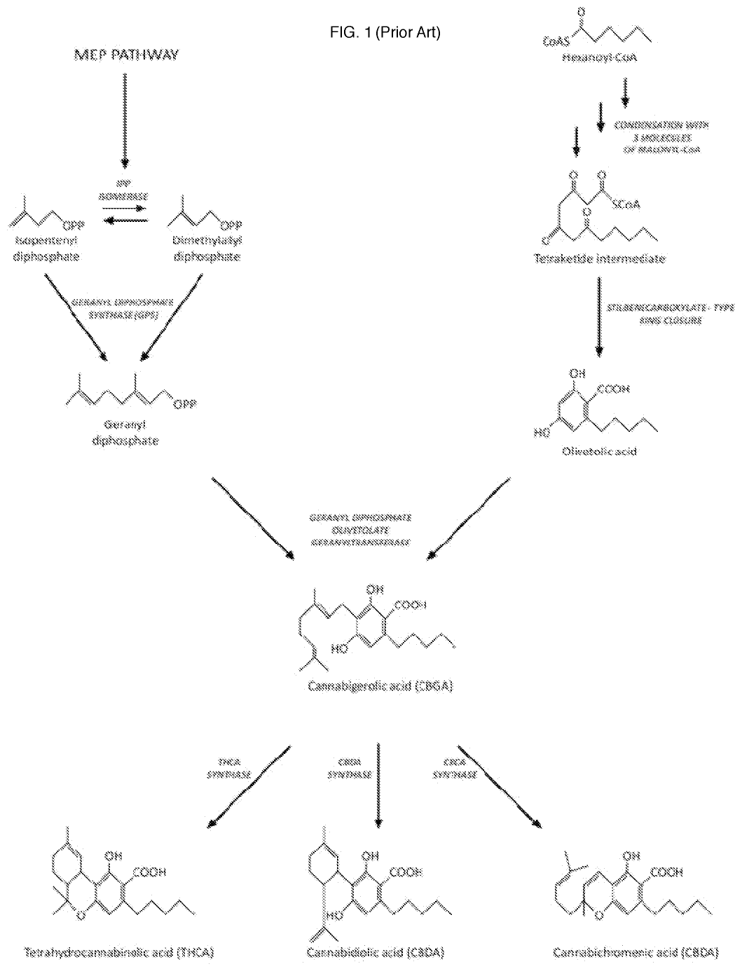

The cannabinoid pathway in cannabis is shown in FIG. 1. As is shown by this Figure, synthesis requires orchestration of several plant enzymes and two separate pathways in order to produce the cannabinoid end products.

Many algae species are capable of overproducing triglycerides and free fatty acids. The fatty acid synthesis and the terpenoids synthesis pathways share some of the initial building blocks with the pathway of the phyto-cannabinoids synthesis. In algae, the whole reservoir of these building blocks is used for fatty acid synthesis.

While reducing the present invention to practice, the present inventor has devised an algae expression system that can be used to produce large amounts of purifiable cannabinoids in algae cultures.

In order to divert the synthesis toward cannabinoid synthesis pathway the present inventor introduced into algae a set of cannabinoid synthesizing enzymes with high affinity toward building blocks and the ability to successfully compete with fatty acid synthesizing enzymes.

There are three important molecules required for cannabinoid synthesis: malonyl-coA, the hexanoyl-coA and geranyl-diphosphate. In algae, in contrast to malonyl-coA and geranyl-diphosphate (GDP), hexanoyl-coA is produced in extremely low quantities. Therefore, endogenous hexanoyl was enriched by introducing into algae a gene for an enzyme that polymerizes Malonyl-coA into Hexanoyl-coA.

Two additional enzymes, olivetolic acid synthase and Olivetolic cyclase, were used to produce olivetolic acid in the hexanoyl-coA enriched cells. Specific prenyl transferase enzyme imported from Cannabis sativa, produced the CBG molecule by prenylation of Olivetolic acid by GDP.

Thus, according to one aspect of the present invention there is provided a method of producing a cannabinoid in algae.

As used herein, the term algae encompasses a polyphyletic group of photosynthetic eukaryotic organisms including unicellular microalgae and cyanobacteria and multicellular algae (e.g., kelp and brown algae). Examples of algae genera that can be used in the present invention include, but are not limited to, Green algae, red algae, Euglenids, Chromista, Dinoflagellates and Cyanobacteria.

The method of the present invention is carried out by expressing in an algae cell an enzyme for converting hexanoic acid to hexanoyl-CoA, enzymes for converting hexanoyl-CoA to olivetolic acid (OA), an enzyme for converting olivetolic acid (OA) to cannabigerolic acid (CbGA) and an enzyme for converting cannabigerolic acid (CbGA) to a cannabinoid.

Such enzymatic activity can be provided by transforming an algae cell with a polynucleotide sequences encoding hexanoyl synthase, prenyl synthase, olivetolic acid cyclase and prenyl transferase.

As is mentioned herein, the present inventor has discovered that while constitutive expression of native polynucleotide sequences can yield detectable amounts of a cannabinoid product, it does not yield commercial amounts of a purifiable product.

Further research has led the present inventor to both alter these sequences to optimize enzyme production in algae and modify expression in order to optimize cannabinoid production.

Genes encoding enzymes, originally from Cannabis sativa plant, are not suitable for translation on algae. To optimize these genes the coding sequences for signal peptides that localized these enzymes in Cannabis organelles such as plastids mitochondria or nucleus were removed. The remaining sequences were optimized for codon usage, specific for each host algae. A similar set of genes carrying added introns in the mRNA were also created. The modified sequences were cloned into available expression vectors such as pSyn_6 for expression in Synechococcus elongatus P7642 or into plasmid pChlamy_4 for expression in Chlamydomonas reinhardtii. Additional vectors included the pOpt2-Venus series of plasmids carrying selective markers Spectinomycin, Paromycin and Hygromycin. All genes in these constructs were expressed constitutively under promotors, PpsB in pSyn_6 plasmid for Synechococcus and Hsp70A promoter in plasmids for expression in Chlamydomonas reinhardtii.

Inducible promoters that can be used in the present invention include, but are not limited to promoters induced by galactose, Isopropyl β-D-1-thiogalactopyranoside (IPTG), heat shock, light or tetracycline.

The transformed algae can be directly exposed to such inducers or they can be added to the culture medium (in the case of chemical inducers); exposure can be timed or not. We constructed inducible vectors pSyn_lacI that carries trc-lac promoter and also lad repressor that blocks transcription. Adding IPTG or lactose to the medium release the transcription and genes are expressed. Using inducible vectors in algae such as Synechococcus elongatus, cannabinoid synthesis is blocked under regular growth conditions and the culture can proliferate without negative effects of cannabinoids on the grows to high cell density. Addition of inducer (IPTG or Lactose) to the culture medium leads to expression of genes resulting in production of large quantities of cannabinoids.

Polynucleotide sequences encoding the enzymes and promoters that can be used by the present invention are listed hereinbelow in the sequence listing section. The polynucleotide sequences encoding the enzymes and promoters can form a part of a polynucleotide construct (expression vector) that is used to transform the algae cells.

Since gene silencing is prevalent in algae, the present polynucleotide sequence can include at least one intron sequence within the coding sequences in order to prevent or minimize such silencing. Coding sequences that include introns are described in the Examples section that follows and are presented in the Sequence Listing.

Several approaches can be used to transform algae cells with the polynucleotide constructs needed to establish a cannabinoid synthesis pathway.

Approaches such as glass beads agitation, electroporation, and microparticle bombardment, have been used to transform C. reinhardtii.

As further described in the Examples section that follows, the present inventor utilized a transformation method that produces 10³ transformants per 1 ug of plasmids DNA.

Briefly, Chlamydomonas cells were prepared for electroporation by a single wash in TAP-sucrose medium and concentrated to 2×10⁸ cells/ml. 30 ng of pure linear plasmids DNA resuspended in water was added to 250 ul of cells and immediately exposed to 800V 25 uF and ∞Ω pulse. Transformed cells were resuspended in 10 ml of TAP sucrose and incubated over night at 26° C. with 200 rpm shaking and in dark. The following day cells were sedimented by centrifugation and plated on TAP agar plates with selective antibiotics.

Synechococcus elongatus cells are capable of DNA uptake from the medium without intervention. Linear DNA molecules are degraded in cells however supercoiled DNA is stable and integrate into the chromosome by homologous recombination. In transformation protocol Synechococcus cells are co-incubated with supercoiled plasmids for 4 hours in 34° C. in dark. Later on cells are centrifuges and plated on BG11-agar plates supplemented with selective antibiotics.

Colonies of transformed algae were isolated streaking on fresh selective agar plates.

Once a stable culture of transformed, enzyme-expressing algae was achieved, the algae was grown in selective medium (TAP for Chlamydomonas reinhardtii or BG11 for Synechococcus elongatus). The growth medium can be supplemented with 10 mM Hexanoic acid and/or 1-10 mM Olivetolic acid in order to increase the amounts of produced cannabinoids.

Crude algae extracts were analyzed to detect CBG and other cannabinoids. The extraction was performed on lyophilized material, cell pellets or the culture medium, using 50% methanol as a solvent and glass beads for mechanical rupture of cell walls and membranes. Aliquots of the crude extract were separated on HPLC reverse phase column with a mass spectrometer detector to identify relevant peaks and purify relevant compounds (e.g., CBD, THC).

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non limiting fashion.

### Example 1: Production of CBG in Chlamydomonas reinhardtii by Constitutive Expression of Cannabis sativa Genes

### Coding Sequence Optimization

Four genes encoding for enzymes: Hexanoyl-CoA synthetase (CsAAE1), Olivetolic acid synthase (B1Q2B6.1), Olivetolic acid cyclase (I6WU39) and Prenyl transferase (CsPt4-T) were codon optimized for expression in Chlamydomonas reinhardtii resulting in sequences SEQ ID NOs: 1-4 respectively.

Subcloning of Genes into Plasmids

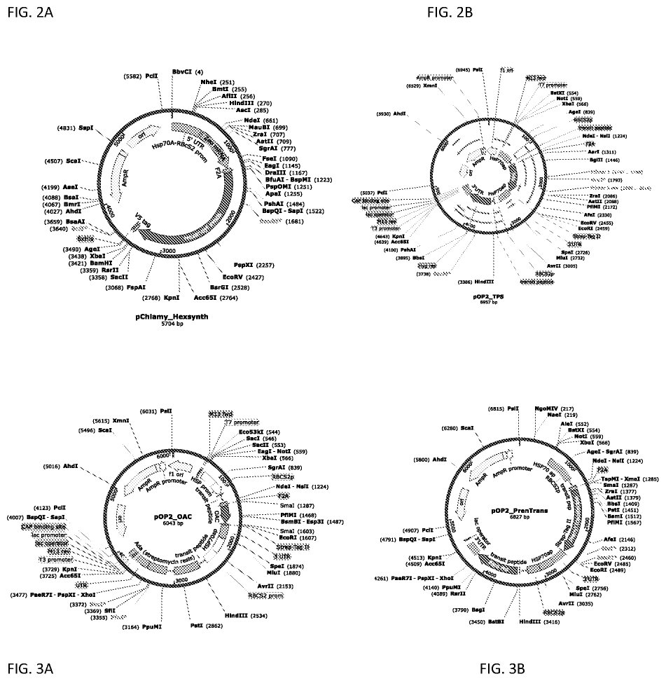

The resulting optimized sequences were cloned into expression plasmids pChlamy_6, pOPt2_ Venus Hyg, pOPt2_ Venus Spe and pOPt2_ Venus Paro respectively. Expression of all genes was regulated by the Heat Shock promoter Hsp70A-RBcS2. Each plasmid carries a selective marker for the antibiotics Zeocin, Hygromycin B, Spectinomycin and Paromomycin (FIGS. 2A-B).

Plasmids were purified and prepared for transformation into Chlamydomonas cells by linearization using AhdI restriction nuclease enzyme.

### Transformation by Electroporation

Chlamydomonas reinhardtii cells were cultured in TAP medium (Difco) under dark/light 12:12 hours regime until they reached logarithmic growth stage (2×10⁶ cells/ml) than were washed with TAP-sucrose medium and concentrated to 2×10⁸ cells/ml. Aliquot of 0.25 ml of competent cells was used for each transformation.

Competent cells were mixed with 30 ng of linear plasmid in prechilled 4 mm electroporation cuvettes. Cuvettes were placed into BioRad Gene Pulser electroporator chamber set at 800V, 25 uF and 000 and an electroporation pulse was delivered to transform cells.

Transformed cells were immediately transferred into 10 ml of TAP-sucrose medium and grown in dark at 25° C. with 200 rpm shaking for 12 hours. Following incubation, cells were precipitated and plated on selective TAP agar plates. Transformed colonies of Chlamydomonas reinhardtii appeared after 8-10 days of incubation at 25° C. with illumination of 50 umol photons/m²/sec.

Chlamydomonas cells were transformed sequentially with all 4 plasmids and every transformation added a new selective marker to the transformed cells.

### Culturing Transformed Clones

Every transformation produced a number of viable clones. Five clones were isolated in every transformation step to overcome the gene silencing phenomenon in Chlamydomonas reinhardtii (silencing of about 50% of the transformed genes).

Isolated clones were cultured in TAP medium containing relevant selective antibiotics. The partially transformed clones that carried only some of the 4 genes, were tested for production of intermediates mainly for olivetolic acid and Hexanoyl-coA formation. Clones that carried all 4 genes were tested for production of CBG.

Cannabinoids Detection

Chlamydomonas reinhardtii clones cultured at 5 or 50 ml medium were centrifuged and the cells pellet as well as the supernatant medium were lyophilized. The dried material was than extracted by 50% methanol the organic phase was analyzed by HPLC using pure marker for:

(i) Malonyl COA

(ii) Hexanoyl COA

(iii) geranyl pyrophosphate (GPP)

(iv) Olivetolic acid

(v) CBGA

(vi) CBG

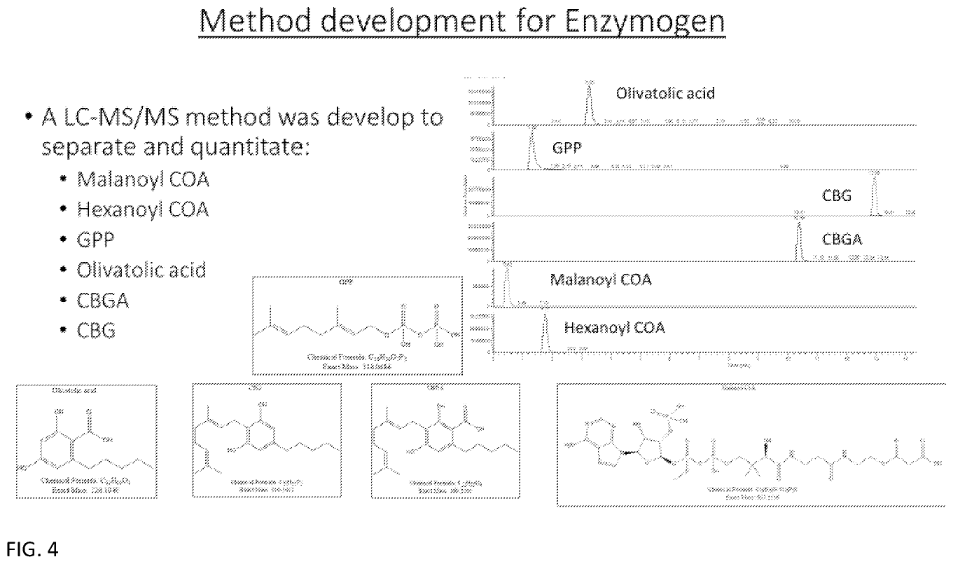

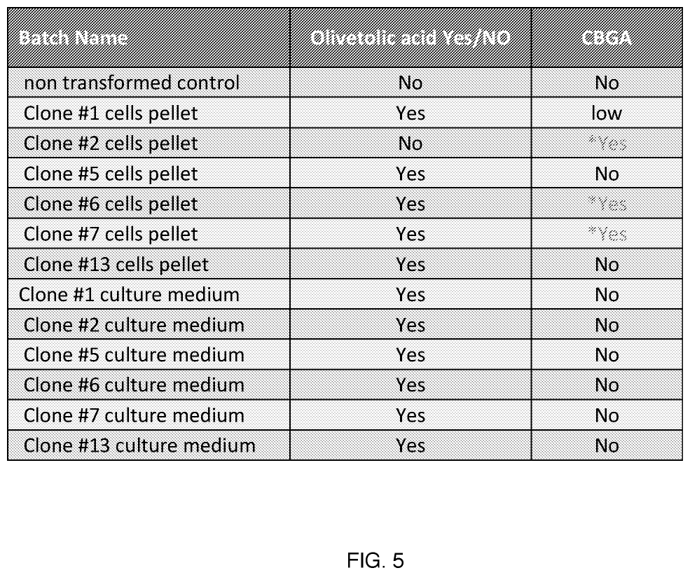

HPLC results are presented in FIGS. 4-5.

### Example 2: Production of CBG in Chlamydomonas reinhardtii Transformed with Genes Carrying Introns

The experiment of Example 2 is similar to that of Example 1 except that the gene sequences were modified by introduction of introns into the coding regions of the 4 enzymes.

Gene silencing is a frequent event in Chlamydomonas reinhardtii. It has been reported that presence of introns in genes significantly improves gene expression of recombinant proteins in Chlamydomonas cells. Hence, 3 of the 4 genes (Prenyl transferase, Olivetolic acid synthetase and Hexanoyl-coA) were modified by adding introns to their coding sequences. A single intron was introduced into the coding sequences of Prenyl transferase and Olivetolic acid synthetase. The gene for Hexanoyl-coA is long therefore 3 introns were added to its coding sequence. Addition of introns resulted in DNA sequences SEQ ID NOs: 5-7 respectively.

### Example 3: Production of CBG in Chlamydomonas reinhardtii by Enzymes Expressed Under Inductive Regulation

The experiment of Example 3 is similar to that of Example 1 with the exception of the regulatory region of the plasmid carrying the gene for Olivetolic acid cyclase. The promoter Hsp70A of the pOPt2-Venus spec was replaced by inducible promoter CYC6. The CBG production process is as described in example 1 however, CBG was not produced due to the absence of Olivetolic acid. CBG production was achieved following addition of Ni to the medium that induced production of Olivetolic acid.

### Example 4: Production of CBG in Synechococcus elongatus by Constitutive Expression of Cannabis sativa Genes

### Genes Optimization

Four genes encoding for enzymes Hexanoyl-CoA synthetase (CsAAE1), Olivetolic acid synthase (B1Q2B6.1), Olivetolic acid cyclase (I6WU39) and Prenyl transferase (CsPt4-4) were codon optimized for expression in Synechcoccus elongatus resulting in sequences SEQ ID NOs: 8-11 respectively.

Subcloning of Genes into Plasmids

The resulting sequences were assembled into single operon with ribosomal binding site attached to each coding region. The whole operon was cloned into expression plasmid pSyn_6. Expression of genes was regulated by the promoter PpsB. The pSyn_cannop plasmid carried selective marker for antibiotics Spectinomycin

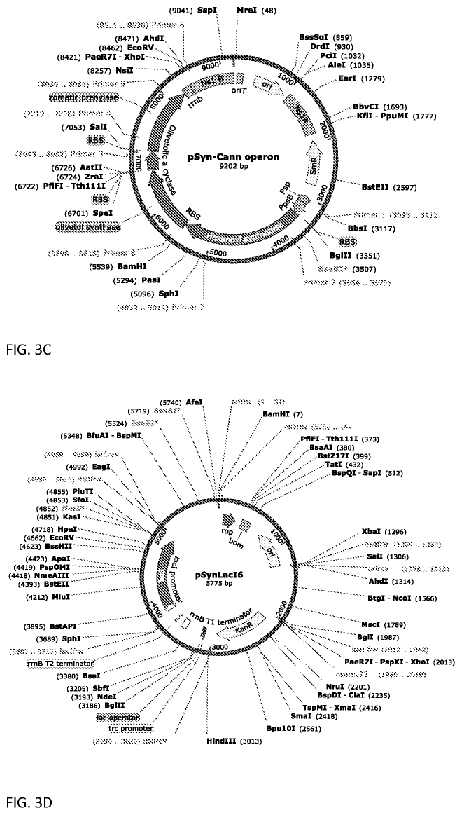

Plasmids (FIGS. 3A-D) were purified and prepared for transformation into Synechcoccus elongatus.

Transformation of Synechococcus elongatus with pSyn_cannop Plasmid

Synechococcus elongatus cells were cultured in BG11 medium (Gibco) at 34° C. under dark/light 12:12 hours regime until they reached logarithmic growth stage (A750=1).1.5 ml of the culture was washed with fresh BG11 medium and finally resuspended in 100 ul of BG11.

The competent cells were mixed with 100 ng of supercoiled plasmid and incubated at 34° C. for 4 hours at dark. Transformed cells were than plated on BG11 agar plates containing 10 ug/ml of Spectinomycin and incubated under illumination of 50 umol photons/m²/sec at 34° C.

### Culturing Transformed Clones

Large number of clones were obtained and 40 clones were analyzed by PCR to detect the 4 gene operon. PCR positive clones were isolated on BG11 agar plates.

Isolated clones were cultured in 5 ml BG11 medium containing the selective antibiotic. Clones that carried all 4 genes were tested for production of CBG.

### Cannabinoids Detection

Synechococcus elongatus clones cultured at 5 or 50 ml medium were centrifuged and the cell pellet as well as the supernatant medium were lyophilized. The dried material was than extracted by 50% methanol the organic phase was analyzed by HPLC using pure marker for:

(i) Malonyl COA

(ii) Hexanoyl COA

(iii) GPP

(iv) Olivetolic acid

(v) CBGA

(vi) CBG

### Example 5: Production of CBG in Synechococcus elongatus by Enzymes Expressed Under Inductive Regulation

The experiment of Example 5 is similar to that of Example 4 with the exception of the regulatory region of the plasmid that carries the 4 gene operon (FIGS. 2A-B).

Plasmid pSyn-lacI that carries a Kanamycin selection marker and a lad repressor was constructed. The promoter (PpsB) was replaced by the inducible promoter trc-lac. The CBG production process is as described in Example 4 however CBG is not produced dues to suppression of the promoter. CBG production is achieved only following addition of inducer such as lactose or IPTG to the medium.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1.-26. (canceled)

27. A method of producing a cannabinoid in algae comprising expressing in an algae cell:
(a) an enzyme for converting hexanoic acid to hexanoyl-CoA;
(b) enzymes for converting hexanoyl-CoA to olivetolic acid (OA);
(c) an enzyme for converting olivetolic acid (OA) to cannabigerolic acid (CbGA); and
(d) an enzyme for converting cannabigerolic acid (CbGA) to a cannabinoid.

28. The method of claim 27, wherein said algae cell is grown in a growth medium.

29. The method of claim 28, wherein said growth medium is supplemented with a supplement selected from the group consisting of hexanoic acid and olivetolic acid.

30. The method of claim 27, wherein said algae cell is transformed with polynucleotide sequences encoding Hexanolyl synthase, Prenyl synthase, Olivetolic acid cyclase and Prenyl transferase.

31. The method of claim 30, wherein said inducible promoter is induced by an inducer selected from the group consisting of galactose, IPTG, Heat shock, light. and tetracycline.

32. The method of claim 28, further comprising recovering said cannabinoid from said growth medium.

33. The method of claim 27, wherein said algae cell further expresses:
(e) an enzyme for converting said cannabinoid to cannabidiol (CBD) or tetrahydrocannabinol (THC).

34. The method of claim 33, wherein said algae cell is further transformed with polynucleotide sequences encoding cannabidiolic acid synthase or THC synthase.

35. The method of claim 30, wherein a sequence of said polynucleotide sequences is optimized for expression in an algae cell.

36. The method of claim 27, wherein said algae cell is selected from the group consisting of Green algae, red algae, Euglenids, Chromista, Dinoflagellates and Cyanobacteria.

37. The method of claim 36, wherein said algae cell is selected from the group consisting of Chlorophytes, Charophyta and Rhodophyta.

38. An expression system for expression in an algae cell comprising polynucleotide sequences encoding Hexanolyl synthase, Prenyl synthase, Olivetolic acid cyclase and Prenyl transferase under the control of an inducible promoter.

39. The expression system of claim 38, wherein said inducible promoter is induced by an inducer selected from the group consisting of galactose, IPTG, Heat shock, light and tetracycline.

40. The expression system of claim 38, wherein a sequence of said polynucleotide sequences is optimized for expression in an algae cell.

41. The expression system of claim 38, wherein said algae cell is selected from the group consisting of Green algae, red algae, Euglenids, Chromista, Dinoflagellates and Cyanobacteria.

42. The expression system of claim 38, wherein said algae cell is selected from the group consisting of Chlorophytes, Charophyta and Rhodophyta.

43. The expression system of claim 38, wherein each of said polynucleotide sequences includes at least one intron.


SEQUENCE LISTING (11 sequences)

SEQ ID NO: 1 - DNA, 2163 bp, Artificial Sequence
  Hexanoyl-CoA synthetase nucleic acid sequence (optimized for expression in Chlamydomonas reinhardtii)
atgggtaagaactacaagagcctcgatagcgtcgtcgcctccgattttatcgcgctgggcatcaccagcgaggtggctgagacgctccatggccgcctggcggagatcgtctgcaactacggggctgctaccccgcagacgtggattaacattgcgaaccatattctgtcccctgatctcccgttttcgctgcatcagatgctcttttacggttgctacaaggactttggtcctgcgcctcccgcgtggattcctgatcccgagaaggtgaagagcaccaacctcggcgccctcctcgagaagcgcggtaaggagtttctcggtgtgaagtacaaggaccctatctcgtcctttagccacttccaggagttcagcgtccggaacccggaggtgtactggcggacggtgctgatggatgagatgaagatctcctttagcaaggacccggagtgcatcctccgccgcgatgacattaacaaccccggtgggtcggagtggctccctggcggctacctcaacagcgcgaagaactgcctcaacgtgaactcgaacaagaagctgaacgacaccatgatcgtctggcgggacgagggcaacgatgatctccccctgaacaagctgacgctcgaccagctccgcaagcgggtctggctggtgggctacgcgctggaggagatgggcctggagaagggctgcgccatcgccatcgacatgccgatgcatgtcgacgccgtggtgatctacctggccatcgtgctggccgggtacgtcgtggtgtcgattgctgacagcttctccgcgcctgagatctccacgcggctgcgcctgagcaaggctaaggccattttcacgcaggatcacattatccgggggaagaagcggattcctctgtactcgcgggtcgtggaggctaagtcgcctatggctattgtgatcccgtgctccgggtcgaacatcggtgctgagctgcgcgatggggacatttcctgggactacttcctcgagcgcgcgaaggagttcaagaactgcgagtttaccgcccgggagcagcctgtggacgcctacaccaacatcctgttctcctcggggacgaccggcgagccgaaggcgatcccgtggacgcaggctacccctctgaaggcggcggcggatgggtggtcgcacctggatatccgcaagggggatgtgattgtgtggccgacgaacctggggtggatgatgggtccttggctcgtgtacgcttccctgctgaacggcgcttccattgcgctgtacaacggctcgcctctggtgagcggttttgcgaagttcgtccaggacgccaaggtcacgatgctgggtgtcgtgccttcgattgtgcggagctggaagtcgacgaactgcgtctccggctacgattggagcaccattcggtgcttctcgtccagcggggaggcgagcaacgtggatgagtacctctggctcatgggtcgggcgaactacaagccggtcatcgagatgtgcggtggtaccgagatcggcggcgcctttagcgcgggctccttcctccaggcgcagtccctgtcgtccttctcgtcgcagtgcatggggtgcacgctctacattctggataagaacggttaccctatgccgaagaacaagccgggtattggggagctcgcgctcggtcctgtgatgttcggtgcgtccaagaccctgctcaacgggaaccaccatgatgtgtactttaaggggatgcctacgctgaacggggaggtcctgcgccggcacggtgatatttttgagctcacctcgaacggctactaccatgcgcatggccgcgcggacgacacgatgaacattggtggcatcaagatttcctccattgagattgagcgcgtctgcaacgaggtggacgatcgggtgttcgagacgaccgccatcggtgtcccgccgctcggtggcggccctgagcagctcgtcattttttttgtcctgaaggactcgaacgatacgaccatcgacctcaaccagctccgcctctcctttaacctcggcctgcagaagaagctgaacccgctgtttaaggtgacccgggtggtcccgctgagctcgctcccgcggaccgccaccaacaagatcatgcgccgggtgctgcgccagcagttcagccattttgagtaa

SEQ ID NO: 2 - DNA, 1158 bp, Artificial Sequence
  Olivetolic acid synthase nucleic acid sequence (optimized for expression in Chlamydomonas reinhardtii)
atgaaccacctgcgcgccgagggccctgcgtccgtgctggctattggcacggccaaccctgagaacattctcctccaggacgagtttcccgattactactttcgggtgacgaagtccgagcacatgacgcagctgaaggagaagttccgcaagatctgcgataagtcgatgatccgcaagcgcaactgctttctcaacgaggagcacctgaagcagaacccgcggctcgtggagcacgagatgcagacgctggatgcccggcaggatatgctcgtggtcgaggtccctaagctggggaaggacgcttgcgctaaggcgattaaggagtgggggcagcctaagtccaagattacccatctgattttcacctccgcttcgaccaccgatatgcccggggccgactaccattgcgctaagctgctcgggctctcgccttcggtcaagcgggtgatgatgtaccagctcggttgctacgggggtggcaccgtgctgcggatcgcgaaggatattgccgagaacaacaagggggcgcgggtcctcgccgtctgctgcgacatcatggcctgcctcttccgcgggccttcggagtccgatctcgagctcctggtgggtcaggctatctttggtgatggggctgcggccgtgattgtcggggctgagccggacgagtccgtgggggagcgccctatcttcgagctcgtgagcacgggtcagacgattctccctaactcggaggggaccattggtggccatattcgggaggccggcctgattttcgatctccacaaggacgtccccatgctgatttccaacaacatcgagaagtgcctcatcgaggcctttacccctatcgggatttcggattggaactccattttttggattacccacccgggcggtaaggctatcctcgacaaggtcgaggagaagctgcacctcaagagcgacaagttcgtggactcccggcacgtcctctccgagcacggtaacatgagctcgagcaccgtgctgtttgtgatggacgagctgcgcaagcgctcgctcgaggagggtaagtccaccaccggcgacggttttgagtggggcgtgctcttcggctttggtcctggcctgacggtggagcgggtggtcgtgcggtcggtgcctattaagtactaa

SEQ ID NO: 3 - DNA, 306 bp, Artificial Sequence
  Olivetolic acid cyclase nucleic acid sequence (optimized for expression in Chlamydomonas reinhardtii)
atggctgtcaagcacctgattgtcctcaagttcaaggacgagatcacggaggcccagaaggaggagttttttaagacctacgtgaacctcgtgaacattatcccggctatgaaggacgtgtactgggggaaggatgtcacgcagaagaacaaggaggagggctacacccacatcgtggaggtcaccttcgagtccgtcgagacgattcaggattacattatccacccggcccacgtcgggttcggcgatgtctaccggtccttttgggagaagctcctgatctttgactacaccccccgcaagtaa

SEQ ID NO: 4 - DNA, 981 bp, Artificial Sequence
  Prenyl transferase amino acid sequence (optimized for expression in Chlamydomonas reinhardtii)
atggctggcagcgatcagattgaggggtcccctcatcatgagtcggataacagcattgcgaccaagatcctcaactttgggcacacgtgctggaagctgcagcgcccctacgtggtgaagggcatgatctcgattgcctgcggcctgtttggtcgggagctctttaacaaccgccatctgttctcctgggggctcatgtggaaggcgttttttgccctcgtgccgatcctcagcttcaacttctttgccgctattatgaaccagatttacgatgtggacatcgaccgcattaacaagcctgatctgccgctggtgagcggggagatgtccattgagaccgcgtggattctctccattatcgtggctctcacgggcctgattgtgacgattaagctgaagtccgctcccctgtttgtctttatctacatcttcggcattttcgctggctttgcttactcggtcccgccgatccggtggaagcagtacccgttcacgaactttctcattacgatttcgtcccacgtgggcctcgcgtttaccagctacagcgccaccacctccgctctcggcctcccgtttgtgtggcggcccgctttctcgttcatcattgccttcatgacggtgatgggtatgacgattgcctttgcgaaggatatctcggatattgagggggacgccaagtacggtgtgagcacggtggctacgaagctcggggctcgcaacatgacgttcgtggtgtccggtgtcctgctgctcaactacctcgtgagcatcagcatcgggatcatttggccccaggtgtttaagtcgaacattatgatcctgtcgcatgctattctcgcgttttgcctcatcttccagacgcgcgagctcgctctcgcgaactacgcctccgccccctcgcggcagttttttgagtttatttggctgctgtactacgccgagtacttcgtctacgtctttatttaagatatcgaattc

SEQ ID NO: 5 - DNA, 1333 bp, Artificial Sequence
  Prenyl transferase nucleic acid sequence (modified by introduction of an intron)
atgggtctctcgtccgtgtgcacgttctccttccagacgaactaccacacgctcctgaacccgcacaacaacaaccccaagacgtcgctcctgtgctaccggcacccgaagaccccgatcaagtacagctacaacaacttcccttccaagcactgcagcaccaagtcctttcatctccagaacaagtgctccgagtcgctgtcgattgcgaagaacagcattcgggctgctaccacgaaccagacggagccgccggagagcgataaccattcggtggcgaccaagatcctgaacttcggtaaggcgtgctggaagctccagcgcccctacacgattatcgcgttcacgtcctgcgcttgcgggctctttgggaaggagctcctgcataacacgaacctcatttcgtggtccctcatgttcaaggctttctttttcctggtggccattctctgcattgcttcgtttacgacgaccatcaaccagatttacgatctccacattgaccggatcaacaagccggatctccccctggccagcggtgagatttccgtcaacacggcgtggatcatgtcgatcatcgtcgcgctgtttgggctcatcatcacgatcaagatgaagggcgggcccctctacatctttgggtactgctttggcattttcggggggatcgtctactcggtgcctccgttccgctggaagcagaacccctcgacggcctttctgctcaacttcctcgcgcacattatcacgaactttacgttttactacgctagccgggctgcgctcggcctcccgttcgagctccgcccctcgtttacgtttctcctggcttttatgaagtcgatgggcagcgctctcgctctcattaaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatgcgagcgatgtggagggtgacacgaagtttggtatcagcacgctcgctagcaagtacggttcgcgcaacctcacgctgttctgctcgggtattgtcctgctcagctacgtggcggccatcctcgccgggattatctggccccaggcctttaacagcaacgtcatgctgctgtcccatgcgattctcgctttttggctcatcctccagacgcgcgattttgctctcaccaactacgatcccgaggctggtcggcggttttacgagtttatgtggaagctctactacgctgagtacctggtctacgtctttatctaa

SEQ ID NO: 6 - DNA, 1303 bp, Artificial Sequence
  Olivetolic acid synthetase nucleic acid sequence (modified by introduction of an intron)
atgaaccacctgcgcgccgagggccctgcgtccgtgctggctattggcacggccaaccctgagaacattctcctccaggacgagtttcccgattactactttcgggtgacgaagtccgagcacatgacgcagctgaaggagaagttccgcaagatctgcgataagtcgatgatccgcaagcgcaactgctttctcaacgaggagcacctgaagcagaacccgcggctcgtggagcacgagatgcagacgctggatgcccggcaggatatgctcgtggtcgaggtccctaagctggggaaggacgcttgcgctaaggcgattaaggagtgggggcagcctaagtccaagattacccatctgattttcacctccgcttcgaccaccgatatgcccggggccgactaccattgcgctaagctgctcgggctctcgccttcggtcaagcgggtgatgatgtaccagctcggttgctacgggggtggcaccgtgctgcggatcgcgaaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggatattgccgagaacaacaagggggcgcgggtcctcgccgtctgctgcgacatcatggcctgcctcttccgcgggccttcggagtccgatctcgagctcctggtgggtcaggctatctttggtgatggggctgcggccgtgattgtcggggctgagccggacgagtccgtgggggagcgccctatcttcgagctcgtgagcacgggtcagacgattctccctaactcggaggggaccattggtggccatattcgggaggccggcctgattttcgatctccacaaggacgtccccatgctgatttccaacaacatcgagaagtgcctcatcgaggcctttacccctatcgggatttcggattggaactccattttttggattacccacccgggcggtaaggctatcctcgacaaggtcgaggagaagctgcacctcaagagcgacaagttcgtggactcccggcacgtcctctccgagcacggtaacatgagctcgagcaccgtgctgtttgtgatggacgagctgcgcaagcgctcgctcgaggagggtaagtccaccaccggcgacggttttgagtggggcgtgctcttcggctttggtcctggcctgacggtggagcgggtggtcgtgcggtcggtgcctattaagtactaa

SEQ ID NO: 7 - DNA, 2453 bp, Artificial Sequence
  Hexanoyl-coA synthetase nucleic acid sequence (modified by introduction of 3 introns)
atgggtaagaactacaagagcctcgatagcgtcgtcgcctccgattttatcgcgctgggcatcaccagcgaggtggctgagacgctccatggccgcctggcggagatcgtctgcaactacggggctgctaccccgcagacgtggattaacattgcgaaccatattctgtcccctgatctcccgttttcgctgcatcagatgctcttttacggttgctacaaggactttggtcctgcgcctcccgcgtggattcctgatcccgagaaggtgaagagcaccaacctcggcgccctcctcgagaagcgcggtaaggagtttctcggtgtgaagtacaaggaccctatctcgtcctttagccacttccaggagttcagcgtccggaacccggaggtgtactggcggacggtgctgatggatgagatgaagatctcctttagcaaggacccggagtgcatcctccgccgcgatgacattaacaaccccggtgggtcggagtggctccctggcggctacctcaacagcgcgaagaactgcctcaacgtgaactcgaacaagaagctgaacgacaccatgatcgtctggcgggacgagggcaacgatgatctccccctgaacaagctgacgctcgaccagctccgcaagcgggtctggctggtgggctacgcgctggaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggagatgggcctggagaagggctgcgccatcgccatcgacatgccgatgcatgtcgacgccgtggtgatctacctggccatcgtgctggccgggtacgtcgtggtgtcgattgctgacagcttctccgcgcctgagatctccacgcggctgcgcctgagcaaggctaaggccattttcacgcaggatcacattatccgggggaagaagcggattcctctgtactcgcgggtcgtggaggctaagtcgcctatggctattgtgatcccgtgctccgggtcgaacatcggtgctgagctgcgcgatggggacatttcctgggactacttcctcgagcgcgcgaaggagttcaagaactgcgagtttaccgcccgggagcagcctgtggacgcctacaccaacatcctgttctcctcggggacgaccggcgagccgaaggcgatcccgtggacgcaggctacccctctgaaggcggcggcggatgggtggtcgcacctggatatccgcaagggggatgtgattgtgtggccgacgaacctggggtggatgatgggtccttggctcgtgtacgcttccctgctgaacggcgcttccattgcgctgtacaacggctcgcctctggtgagcggttttgcgaagttcgtccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggacgccaaggtcacgatgctgggtgtcgtgccttcgattgtgcggagctggaagtcgacgaactgcgtctccggctacgattggagcaccattcggtgcttctcgtccagcggggaggcgagcaacgtggatgagtacctctggctcatgggtcgggcgaactacaagccggtcatcgagatgtgcggtggtaccgagatcggcggcgcctttagcgcgggctccttcctccaggcgcagtccctgtcgtccttctcgtcgcagtgcatggggtgcacgctctacattctggataagaacggttaccctatgccgaagaacaagccgggtattggggagctcgcgctcggtcctgtgatgttcggtgcgtccaagaccctgctcaacgggaaccaccatgatgtgtactttaaggggatgcctacgctgaacggggaggtcctgcgccggcacggtgatatttttgagctcacctcgaacggctactaccatgcgcatggccgcgcggacgacacgatgaacattggtggcatcaagatttcctccattgagattgagcgcgtctgcaacgaggtggacgatcgggtgttcgagacgaccgccatcggtgtcccgccgctcggtggcggccctgagcagctcgtcattttttttgtcctgaaggactcgaacgatacgaccatcgacctcaaccagctccgcctctcctttaacctcggcctgcagaagaagctgaacccgctgtttaaggtgacccgggtggtcccgctgagctcgctcccgcggaccgccaccaacaagatcatgcgccgggtgctgcgccagcagttcagccattttgagtaa

SEQ ID NO: 8 - DNA, 2453 bp, Artificial Sequence; identical to SEQ ID NO: 7
  Hexanoyl-CoA synthetase nucleic acid sequence (optimized for expression in Synechcoccus elongatus)

SEQ ID NO: 9 - DNA, 1158 bp, Artificial Sequence
  Olivetolic acid synthase nucleic acid sequence (optimized for expression in Synechcoccus elongatus)
atgaaccacctgcgcgctgaaggccccgctagcgtgctggctatcggcaccgctaaccccgaaaacatcctgctgcaagacgaattccccgactactacttccgcgtgaccaaaagcgaacacatgacccaactgaaagaaaaattccgcaaaatctgcgacaaaagcatgatccgcaaacgcaactgcttcctgaacgaagaacacctgaaacaaaacccccgcctggtggaacacgaaatgcaaaccctggacgctcgccaagacatgctggtggtggaagtgcccaaactgggcaaagacgcttgcgctaaagctatcaaagaatggggccaacccaaaagcaaaatcacccacctgatcttcaccagcgctagcaccaccgacatgcccggcgctgactaccactgcgctaaactgctgggcctgagccccagcgtgaaacgcgtgatgatgtaccaactgggctgctacggcggcggcaccgtgctgcgcatcgctaaagacatcgctgaaaacaacaaaggcgctcgcgtgctggctgtgtgctgcgacatcatggcttgcctgttccgcggccccagcgaaagcgacctggaactgctggtgggccaagctatcttcggcgacggcgctgctgctgtgatcgtgggcgctgaacccgacgaaagcgtgggcgaacgccccatcttcgaactggtgagcaccggccaaaccatcctgcccaacagcgaaggcaccatcggcggccacatccgcgaagctggcctgatcttcgacctgcacaaagacgtgcccatgctgatcagcaacaacatcgaaaaatgcctgatcgaagctttcacccccatcggcatcagcgactggaacagcatcttctggatcacccaccccggcggcaaagctatcctggacaaagtggaagaaaaactgcacctgaaaagcgacaaattcgtggacagccgccacgtgctgagcgaacacggcaacatgagcagcagcaccgtgctgttcgtgatggacgaactgcgcaaacgcagcctggaagaaggcaaaagcaccaccggcgacggcttcgaatggggcgtgctgttcggcttcggccccggcctgaccgtggaacgcgtggtggtgcgcagcgtgcccatcaaatactag

SEQ ID NO: 10 - DNA, 306 bp, Artificial Sequence
  Olivetolic acid cyclase nucleic acid sequence (optimized for expression in Synechcoccus elongatus)
atggctgtgaaacacctgatcgtgctgaaattcaaagacgaaatcaccgaagctcaaaaagaagaattcttcaaaacctacgtgaacctggtgaacatcatccccgctatgaaagacgtgtactggggcaaagacgtgacccaaaaaaacaaagaagaaggctacacccacatcgtggaagtgaccttcgaaagcgtggaaaccatccaagactacatcatccaccccgctcacgtgggcttcggcgacgtgtaccgcagcttctgggaaaaactgctgatcttcgactacaccccccgcaaatag

SEQ ID NO: 11 - DNA, 978 bp, Artificial Sequence
  Prenyl transferase nucleic acid sequence (optimized for expression in Synechcoccus elongatus)
atggcagggtcggaccagatcgaaggctcccctcaccacgaatccgataacagtattgcaacaaagattttgaatttcggccatacatgttggaaactgcaacggccatacgtcgtcaaggggatgatctcgattgcctgcggcctgtttgggcgtgaactgttcaacaatcgccacttgttcagttggggtctgatgtggaaggcgttcttcgcactcgtcccgatcttgtcgtttaattttttcgcggcaattatgaaccagatttacgatgtggatattgatcgtattaacaaacccgacttgccactggtcagtggtgaaatgagcatcgagacggcgtggattctctccatcatcgtcgcgctgactggcttgattgtcacaatcaaactgaaatccgcacccctctttgtgttcatctacatcttcggaatctttgcaggcttcgcgtattcggtcccgccgattcgttggaaacagtatccgttcacgaatttcctgattacgattagtagtcacgttggtctggctttcacttcctattccgctaccaccagcgcgttggggttgccttttgtctggcggcccgccttcagcttcattatcgcatttatgacagtcatgggcatgactattgcattcgctaaggacatcagcgacattgagggagacgcaaagtacggtgtgtccaccgttgcaactaaactcggggcacggaatatgacatttgtcgtcagcggagtgctgctgctcaattatttggtctcgattagtattggcatcatttggccacaggtgtttaagtcgaacattatgattttgagtcatgccatcctcgcattctgcctcatctttcagactcgcgagctggcattggccaactacgcaagcgcaccctcccgccaattctttgagtttatttggctcctctactacgctgaatattttgtctatgtgtttatctagtaaatgcat